(19) United States
(12) Patent Application Publication
(10) Pub. No.: US 2003/0143629 A1
(43) Pub. Date: Jul. 31, 2003

(54) Compositions and methods of production and use of polydiazoaminotyrosine

(76) Inventor: Eric A. Holwitt, San Antonio, TX (US)

Correspondence Address: Blakely, Sokoloff, Taylor & Zafman, Seventh Floor, 12400 Wilshire Boulevard, Los Angeles, CA, 90025-1030, US

(21) Appl. No.: 10/310,553

(22) Filed: Dec. 4, 2002

Related U.S. Application Data
(60) Provisional application No. 60/344,502, filed on Dec. 28, 2001.

(51) Int. Cl. G01N 33/53; A61K 31/655; C09B 29/36

(52) U.S. Cl. 435/7.1; 534/798; 514/150

(57) ABSTRACT

The present invention concerns compositions, methods of production and methods of use of polydiazoaminotyrosine (DAT), a novel organic semiconductor. In preferred embodiments, the DAT is oxidized (O-DAT). In certain embodiments, recognition complexes comprising DAT operably coupled to a binding moiety are provided. The recognition complexes are of use for detection, identification and/or neutralization of various analytes. In alternative embodiments, DAT in combination with a source of activating radiation may be used to neutralize various analytes, such as anthrax spores.

### RELATED APPLICATIONS

The present application claims the benefit under 35 U.S.C. §119(e) of provisional patent application serial No. 60/344,502, filed Dec. 28, 2001.

### FEDERALLY SPONSORED RESEARCH OF DEVELOPMENT

The invention described herein is made with Government support under contract F41624-00-D-7000-01 awarded by the Department of the Air Force. The Federal Government has rights in the invention.

### BACKGROUND OF THE INVENTION

1. Field of the Invention

The present invention relates to the field of organic semiconductors. More particularly, the invention relates to compositions, methods of production and methods of use of polydiazoaminotyrosine (“DAT”). In certain embodiments, DAT alone or in combination with various binding moieties is of use for the detection, identification and/or neutralization of various analytes.

2. Description of Related Art

Organic semiconductors are conjugated organic compounds in which electrons or electron “holes” can move through regions of the conjugated system that vary in nature from insulator to conductor. An organic semiconductor may be thought of as the organic equivalent of a metal in terms of electrical properties. Organic semiconductors differ substantially from metals in their spectroscopic properties, which may include fluorescence and/or luminescence. Organic semiconductors may be characterized by their absorption, reflection or emission of electromagnetic radiation, including infrared, ultraviolet or visible light

An example of an organic semiconductor is diazoluminomelanin (DALM) (U.S. Pat. Nos. 5,003,050, 5,156,971, 5,856,108 and 5,902,728, each incorporated herein by reference). DALM exhibits spectroscopic and energy transducing properties that are of use for analyte detection, identification and neutralization (U.S. Pat. No. 6,303,316). The manufacture of DALM is a complicated process that results in the generation of organic solvent waste. A need exists for an organic semiconductor with the spectroscopic properties of DALM, but with increased ease of production and/or use.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a binding moiety-organic semiconductor couplet system in accordance with an exemplary embodiment of the present invention.

FIG. 2 illustrates another exemplary embodiment of a binding moiety-organic semiconductor couplet system, using binding moiety-organic semiconductor couplets attached to magnetic beads. The flow chart illustrates the operational relationships between the components of a binding moiety-organic semiconductor couplet system.

FIG. 3A shows the excitation and emission profile of DALM

FIG. 3B shows the excitation and emission profile of DAT.

FIG. 4A shows the emission profile of magnetic beads plus DNA, with or without DAT added.

FIG. 4B shows the emission profile of magnetic beads plus DAT, with or without DNA added.

### DESCRIPTION OF ILLUSTRATIVE EMBODIMENTS

Definitions

As used herein, “a” or “an” may mean one or more than one of an item.

“Organic semiconductor” means a conjugated (alternating double and single bonded) organic compound in which regions of electrons and the absence of electrons (holes or positive charges) can move with varying degrees of difficulty through the aligned conjugated system (varying from insulator to conductor). An organic semiconductor may be thought of as the organic equivalent of a metal, in terms of electrical properties. Organic semiconductors are distinguished from metals in their spectroscopic properties. Organic semiconductors of use in the practice of the instant invention may be fluorescent, luminescent, chemiluminescent, sonochemiluminescent, thermochemiluminescent or electrochemiluminescent (Bruno et al., 1998) or may be otherwise characterized by their absorption, reflection or emission of electromagnetic radiation, including infrared, ultraviolet or visible light. In preferred embodiments, the organic semiconductor is DAT.

“Binding moiety” refers to a molecule or aggregate of molecules that has a binding affinity for one or more analytes. The term is not limiting as to the type of molecule or aggregate. Non-limiting examples of binding moieties include peptides, polypeptides, proteins, glycoproteins, antibodies, antibody fragments, antibody derivatives, receptors, enzymes, transporters, binding proteins, cytokines, hormones, substrates, substrate analogs, metabolites, inhibitors, activators, lipids, glycolipids, carbohydrates, polysaccharides, nucleic acids, nucleic acid ligands, polynucleotides and oligonucleotides, as well as chemically modified forms of each.

“Binding” refers to an interaction between a target and a binding moiety, resulting in a sufficiently stable complex so as to permit separation of complexes from uncomplexed molecules under given binding or reaction conditions. Binding may be mediated through covalent bonding, hydrogen bonding, ionic bonding, Van der Waals interactions, hydrophobic interactions or other molecular forces.

“Analyte,” “target” and “agent” are used herein synonymously to mean any compound or aggregate of interest for detection, identification and/or neutralization. Non-limiting examples of analytes include a protein, peptide, carbohydrate, polysaccharide, glycoprotein, nucleic acid, lipid, hormone, receptor, antigen, allergen, antibody, substrate, metabolite, cofactor, inhibitor, drug, pharmaceutical, nutrient, toxin, poison, explosive, pesticide, chemical warfare agent, biohazardous agent, prion, radioisotope, vitamin, heterocyclic aromatic compound, carcinogen, mutagen, narcotic, amphetamine, barbiturate, hallucinogen, waste product, contaminant or other molecule. Molecules of any size can serve as targets. Analytes are not limited to single molecules, but may also comprise complex aggregates of molecules, such as a virus, bacterium, spore, anthrax spore, mold, yeast, algae, amoebae, Ghiardia, dinoflagellate, unicellular organism, pathogen, cell or infectious agent. In certain embodiments, cells exhibiting a particular characteristic or disease state, such as a cancer cell, may be target analytes. Virtually any chemical or biological effector would be a suitable target.

Non-limiting examples of infectious agents within the meaning of “analyte” include those listed in Table 1.

TABLE 1
Infectious Agents

Actinobacillus spp.
Actinomyces spp.
Adenovirus (types 1, 2, 3, 4, 5 et 7)
Adenovirus (types 40 and 41)
Aerococcus spp.
Aeromonas hydrophila
Ancylostoma duodenale
Angiostrongylus cantonensis
Ascaris lumbricoides
Ascaris spp.
Aspergillus spp.
Bacillus anthracis
Bacillus cereus
Bacteroides spp.
Balantidium coli
Bartonella bacilliformis
Blastomyces dermatitidis
Bluetongue virus
Bordetella bronchiseptica
Bordetella pertussis
Borrelia burgdorferi
Branhamella catarrhalis
Brucella spp.
B. abortus
B. canis,
B. melitensis
B. suis
Brugia spp.
Burkholderia mallei
Burkholderia pseudomallei
Campylobacter fetus subsp. fetus
Campylobacter jejuni
C. coli
C. fetus subsp. jejuni
Candida albicans
Capnocytophaga spp.
Chlamydia psittaci
Chlamydia trachomatis
Citrobacter spp.
Clonorchis sinensis
Clostridium botulinum
Clostridium difficile
Clostridium perfringens
Clostridium tetani
Clostridium spp.
Coccidioides immitis
Colorado tick fever virus
Corynebacterium diphtheriae
Coxiella burnetii
Coxsackievirus
Creutzfeldt-Jakob agent, Kuru agent
Crimean-Congo hemorrhagic fever virus
Cryptococcus neoformans
Cryptosporidium parvum
Cytomegalovirus
Dengue virus (1, 2, 3, 4)
Diphtheroids
Eastern (Western) equine encephalitis virus
Ebola virus
Echinococcus granulosus
Echinococcus multilocularis
Echovirus
Edwardsiella tarda
Entamoeba histolytica
Enterobacter spp.
Enterovirus 70
Epidermophyton floccosum,
Microsporum spp. Trichophyton spp.
Epstein-Barr virus
Escherichia coli, enterohemorrhagic
Escherichia coli, enteroinvasive
Escherichia coli, enteropathogenic
Escherichia coli, enterotoxigenic
Fasciola hepatica
Francisella tularensis
Fusobacterium spp.
Gemella haemolysans
Giardia lamblia
Giardia spp.
Haemophilus ducreyi
Haemophilus influenzae (group b)
Hantavirus
Hepatitis A virus
Hepatitis B virus
Hepatitis C virus
Hepatitis D virus
Hepatitis E virus
Herpes simplex virus
Herpesvirus simiae
Histoplasma capsulatum
Human coronavirus
Human immunodeficiency virus
Human papillomavirus
Human rotavirus
Human T-lymphotrophic virus
Influenza virus
Junin virus/Machupo virus
Klebsiella spp.
Kyasanur Forest disease virus
Lactobacillus spp.
Legionella pneumophila
Leishmania spp.
Leptospira interrogans
Listeria monocytogenes
Lymphocytic choriomeningitis virus
Marburg virus
Measles virus
Micrococcus spp.
Moraxella spp.
Mycobacterium spp.
Mycobacterium tuberculosis, M. bovis
Mycoplasma hominis, M. orale,
M. salivarium, M. fermentans
Mycoplasma pneumoniae
Naegleria fowleri
Necator americanus
Neisseria gonorrhoeae
Neisseria meningitidis
Neisseria spp.
Nocardia spp.
Norwalk virus
Omsk hemorrhagic fever virus
Onchocerca volvulus
Opisthorchis spp.
Parvovirus B19
Pasteurella spp.
Peptococcus spp.
Peptostreptococcus spp.
Plesiomonas shigelloides
Powassan encephalitis virus
Proteus spp.
Pseudomonas spp.
Rabies virus
Respiratory syncytial virus
Rhinovirus
Rickettsia akari
Rickettsia prowazekii, R. canada
Rickettsia rickettsii
Ross river virus/O'Nyong-Nyong virus
Rubella virus
Salmonella choleraesuis
Salmonella paratyphi
Salmonella typhi
Salmonella spp.
Schistosoma spp.
Scrapie agent
Serratia spp.
Shigella spp.
Sindbis virus
Sporothrix schenckii
St. Louis encephalitis virus
Murray Valley encephalitis virus
Staphylococcus aureus
Streptobacillus moniliformis
Streptococcus agalactiae
Streptococcus faecalis
Streptococcus pneumoniae
Streptococcus pyogenes
Streptococcus salivarius
Taenia saginata
Taenia solium
Toxocara canis, T. cati
Toxoplasma gondii
Treponema pallidum
Trichinella spp.
Trichomonas vaginalis
Trichuris trichiura
Trypanosoma brucei
Ureaplasma urealyticum
Vaccinia virus
Varicella-zoster virus
Venezuelan equine encephalitis
Vesicular stomatitis virus
Vibrio cholerae, serovar 01
Vibrio parahaemolyticus
Wuchereria bancrofti
Yellow fever virus
Yersinia enterocolitica
Yersinia pseudotuberculosis
Yersinia pestis

“Binding moiety-organic semiconductor couplet” and “couplet” refer to a binding moiety that is operably coupled to an organic semiconductor, preferably DAT. “Operably coupled” means that the binding moiety and the organic semiconductor are in close physical proximity to each other, such that binding of an analyte to the binding moiety results in a change in the properties of the organic semiconductor that is detectable as a signal. In preferred embodiments, the signal is a photochemical signal, such as a fluorescent signal, a luminescent signal, a phosphorescent signal or a change of color. In one embodiment, the signal is a change in the fluorescence emission profile of the binding moiety-organic semiconductor couplet. Operable coupling may be accomplished by a variety of interactions, including but not limited non-covalent or covalent binding of the organic semiconductor to the binding moiety. In another embodiment, the binding moiety may be at least partially embedded in the organic semiconductor. Virtually any type of interaction between the organic semiconductor and the binding moiety is contemplated within the scope of the present invention, so long as the binding of an analyte to the binding moiety results in a change in the properties of the organic semiconductor.

“Photochemical” means any light related process. A “photochemical signal” includes, but is not limited to, a fluorescent signal, a luminescent signal or a change of color.

A “recognition complex” comprises a binding moiety that is operably coupled to an organic semiconductor, such as DAT. A “recognition complex system” comprises an array of recognition complexes. In certain embodiments, the array of recognition complexes is operably coupled to a detection unit, such that changes in the photochemical properties of the organic semiconductor that result from binding of analyte to binding moiety may be detected by the detection unit.

“Nucleic acid ligand” means a non-naturally occurring nucleic acid having a desirable action on a target. A desirable action includes, but is not limited to, binding of the target, catalytically changing the target, reacting with the target in a way that modifies or alters the target or the functional activity of the target, covalently attaching to the target, facilitating the reaction between the target and another molecule, and neutralizing the target. In a preferred embodiment, the action is specific binding affinity for a target molecule, such target molecule being a three dimensional chemical structure.

DAT

In preferred embodiments, the organic semiconductor of use in the disclosed compositions, methods and apparatus is DAT. DAT is a novel organic semiconductor whose production and use are disclosed for the first time herein. Additional details on the production of DAT are provided in Example 1 below. The skilled artisan will realize that the compositions, methods and apparatus of the claimed invention are not limited to the specific embodiments disclosed herein, but also encompass various modifications and/or substitutions.

Generally, DAT may be produced by reacting 3-amino-L-tyrosine (3AT), with an alkali metal nitrite, such as NaNO₂. In preferred embodiments, the 3AT is dissolved first in an aqueous or similar medium before reaction with NaNO₂. Surprisingly, the product of this reaction exhibits spectroscopic properties similar to DALM (U.S. Pat. No. 6,303,316). DALM is synthesized using luminol, a known luminescent compound. It was unexpected that DAT synthesized without incorporation of any luminol would show luminescent characteristics similar to DALM.

Since diazotization reactions are, in general, exothermic, in some embodiments the reaction may be carried out under isothermal conditions or at a reduced temperature, such as, for example, at ice bath temperatures. The reaction may be carried out with refluxing for 1 hour, 2 hours, 4 hours, 6 hours or preferably 8 hours, although longer reaction periods of 10, 12, 14, 18, 20 or even 24 hours are contemplated.

The DAT may be precipitated from aqueous solution by addition of a solvent in which DAT is not soluble, such as acetone. After centrifuging the precipitate and discarding the supernatant, the solid material may be dried under vacuum.

In general, the quantities of the 3AT and alkali metal nitrite reactants used are equimolar. It is, however, within the scope of the invention to vary the quantities of the reactants. The molar ratio of 3AT:metal nitrite may be varied over the range of about 0.6:1 to 3:1.

In alternative embodiments, DAT may be partially or fully oxidized prior to use, resulting in the production of oxidized-DAT (O-DAT). Reduced DAT is dissolved in 5 ml of distilled water with 0.2 gm of sodium bicarbonate added. Five milliliters of 30% hydrogen peroxide is added and the mixture is refluxed until the color of the solution changes from brown to yellow. The mixture is cooled, dialyzed against distilled water and lyophilized. The lyophilized powder contains O-DAT.

In certain embodiments, an organic semiconductor such as DAT may be used to neutralize various agents, including but not limited to anthrax spores (Kiel et al., 1999a, 1999b). The energy transducing properties of organic semiconductors facilitate the inactivation of agents by microwaves, visible light, ultraviolet, infrared or radiofrequency irradiation or exposure to pulsed corona radiation (Titan Industries, San Diego, Calif.). Although the precise mechanism by which organic semiconductors facilitate agent inactivation is unknown, it is possible that the organic semiconductor can absorb various types of radiation and convert it to heat, resulting in explosive heating of membrane bound agents or in thermal denaturation of non-membrane bound agents.

In alternative embodiments, binding moieties that bind to an analyte with high affinity can be produced for use to inactivate or destroy the analyte. A high affinity binding moiety may be attached to an organic semiconductor, such as DAT. The DAT/binding moiety couplet, after binding to the analyte, may be activated by a variety of techniques, including exposure to sunlight, heat, or irradiation of various types, including laser, microwave, radiofrequency, ultraviolet, pulsed corona and infrared. Activation of the DAT/binding moiety couplet results in absorption of energy, which may be transmitted to the analyte, inactivating or destroying it.

In other embodiments, organic semiconductors such as DAT may be operably coupled to one or more binding moieties and used to detect analytes. In such embodiments, binding of analyte to the organic semiconductor:binding moiety couplet may result in a change in the photochemical properties of the couplet that is detectable, for example, as a change in the light emission spectrum of the couplet.

Recognition Complex System

A recognition complex system comprises an array of recognition complexes, each recognition complex comprising a binding moiety. In various embodiments, the binding moiety may be attached to an organic semiconductor, such as DAT. In certain embodiments, the recognition complexes are arranged in a two-dimensional array that may be attached to a glass or other flat surface. In other embodiments, the recognition complexes comprise binding moieties attached to magnetic beads or to glass or polystyrene beads in a three-dimensional array. In a preferred embodiment, the beads are suspended in a liquid medium.

The array of recognition complexes is exposed to analyte. Binding of analyte to individual recognition complexes is detected, for example, by changes in the photochemical properties of the recognition complex upon binding to the analyte. Where the recognition complexes comprise an organic semiconductor, such as DAT, the changes in photochemical properties may be detected by a variety of techniques, described in detail below.

In certain embodiments, an iterative process may be used to increase the specificity of the array of recognition complexes for the analyte. In each round of iteration, the array is exposed to the analyte. Recognition complexes that bind to the analyte are separated from recognition complexes that do not bind to the analyte. Methods for separating bound from unbound recognition complexes are described below. The binding moieties from recognition complexes that bind to the analyte are selected and used to make a new array of recognition complexes. The new array will contain a higher proportion of recognition complexes that bind to the analyte, producing a stronger and more specific photochemical signal. This iterative process may be used to select binding moieties that bind to the analyte with high affinity. Such high affinity binding moieties will be useful in numerous applications, described below. One such application involves production of a neutralizing agent that can inactivate or destroy the target analyte.

Embodiments Involving a Chip Type of Array

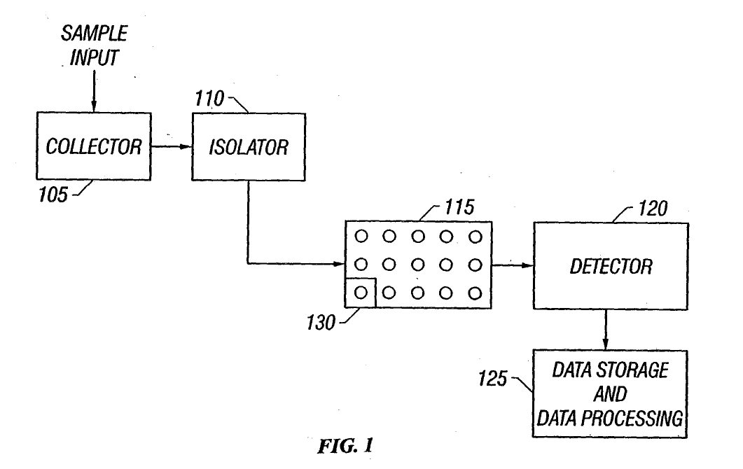

FIG. 1 illustrates a recognition complex system in accordance with a non-limiting exemplary embodiment. This embodiment of the recognition complex system includes a sample collection unit 105, an analyte isolation unit 110, an organic semiconductor chip based array of recognition complexes 115, a detection unit 120 and a data storage and processing unit 125. In general, the sample collection unit 105 is employed to actively collect or passively receive samples containing the unknown analyte to be identified. The analyte isolation unit 110 is employed to filter the sample and isolate the unknown analyte from other substances or compounds that might be present in the sample. The sample collection unit 105 and the analyte isolation unit 110 may be implemented in accordance with any number of known techniques and/or components known in the art.

The array of recognition complexes 115 comprises one or more individual recognition complexes 130. It will be understood that the array of recognition complexes 115 is shown as comprising fifteen recognition complexes for illustrative purposes only. In actuality, the array 115 may contain significantly more than fifteen recognition complexes. Within the scope of the invention, the array may comprise approximately 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 125, 130, 140, 150, 160, 170, 175, 180, 185, 190, 200, 225, 250, 275, 300, 325, 350, 375, 400, 425, 450, 475, 500, 550, 600, 650, 700, 750, 800, 850, 900, 950, 1000, 1500, 2000, 2500, 3000, 3500, 4000, 4500, 5000, 6000, 7000, 8000, 9000, 10000, 15000, 20000, 30000, 40000, 50000, 75000, 10000, 20000, 30000, 40000, 50000, 100000, 200000, 500000, 10⁶, 10⁷, 10⁸, 10⁹, 10¹⁰, 10¹¹, 10¹², 10¹⁴, 10¹⁶, 10¹⁸ recognition complexes or any number in between. In certain embodiments, the binding moiety component of each recognition complex differs in sequence from the binding moiety component of the other recognition complexes in the array. In other embodiments, some or all of the binding moieties may be similar or identical in sequence.

Each of the recognition complexes 130 associated with the array 115 comprises a binding moiety/organic semiconductor couplet. In preferred embodiments, the organic semiconductor used is polydiazoaminotyrosine (DAT). In alternative embodiments, the organic semiconductor may be diazoluminomelanin (DALM). However, other organic semiconductors may serve as acceptable substitutes.

As shown in FIG. 1, the recognition complex system comprises an array 115 of recognition complexes, such as recognition complex 130. Each of these recognition complexes comprises a binding moiety/organic semiconductor couplet. Separating each of the recognition complexes is binding material. In some embodiments, the binding moiety sequences may be distributed across the array as a function of charge and size, or alternatively as a function of charge and pI (isoelectric point).

After collecting one or more samples containing the unknown analyte, the analyte is applied to each recognition complex associated with the array 115. In those embodiments where the binding moiety sequences are not identical, some of the binding moieties will exhibit a high affinity for the analyte, some binding moieties will exhibit less affinity for the analyte and some binding moieties will exhibit no affinity for the analyte. The photochemical properties of the binding moiety/organic semiconductor couplet will change depending on the degree to which the binding moieties bind to the analyte. The photochemical properties associated with some recognition complexes will change significantly, while the photochemical properties associated with other recognition complexes may change very little, if at all, upon exposure to a given analyte.

The photochemical changes may involve changes in the color of the binding moiety/organic semiconductor couplet and/or changes in the color intensity. In certain embodiments, the detection unit 120 comprises a charge coupled device (CCD), such as a CCD camera, digital camera, photomultiplier tube or any other functionally equivalent detector.

The photochemical signature of the analyte may consist of a two-dimensional distribution of fluorescence following exposure to long-wavelength ultraviolet light or other excitation. The response of the array 115 at a specific spatial location 130 may be similar for two or more different analytes, but by combining the fluorescence response of many independent measurement locations, specificity can be high. A typical consumer-type CCD-based color video camera has 768×494 discrete detectors. A miniaturized cell utilizing such a camera with an array could have about 380,000 parallel channels (single detectors). Practical considerations would group detectors for lower but less spatially noisy resolution with fewer channels. Hundreds to thousands of channels could easily be achieved. Optimization of the number of channels would minimize channels and thus computational load, while maximizing specificity and classification accuracy.

Analysis of the photochemical signature, by data processing unit 125, may involve a comparison of multiple channels of fluorescence spectral signatures. Comparison of signatures by data processing unit 125 may be implemented using artificial neural networks (such as the Qnet v2000 neural net software package from Vesta Services, Inc., 1001 Green Bay Rd., Winnetka, Ill. 60093), look-up tables or various other decision methods. This would provide a fast comparison of unknown analytes to a database of previously recorded signatures of known analytes. Application of a current flowing through the recognition complexes may result in the enhancement of any photochemical changes that take place as a result of analyte/binding moiety binding, thereby making it easier for the detection unit 120 to detect and quantify those photochemical changes.

In accordance with one aspect of the present invention, unknown chemical and/or biological analytes may be detected and identified in a single, automated binding step, as the reaction between the analyte and the binding moiety sequences distributed across the array 115 produces a relatively unique change in the photochemical properties of the array as a whole. However, where two or more analytes share similar chemical structures, they might cause the array 115 to produce a relatively similar photochemical response.

Thus, in accordance with another aspect of the present invention, a more unique photochemical response from the array 115 may be achieved to more clearly distinguish between structurally similar analytes. To accomplish this, the binding moieties associated with those recognition complexes that bind to the analyte, as indicated by changes in their photochemical properties, may be extracted from the array.

In certain embodiments, individual recognition complexes 130 may be detached from the array 115 by hydrolysis, cleavage, heating or other methods of dissociation at the location of each such recognition complex. The binding moiety sequences exhibiting affinity for analyte may be separated from the analyte by washing the binding moiety bound to analyte with deionized water, salt solutions, detergents, chaotrophic agents, solvents or other solutions that serve to separate the analyte from the binding moiety. The binding moiety sequences that exhibit no affinity for the analyte can be discarded. The extracted binding moiety sequences may be applied to a clean chip to produce a new array 115. Since the new array 115 comprises only those binding moiety sequences that are identified as binding to the analyte, it should exhibit a greater degree of specificity and a higher binding affinity for the analyte.

Once a new array chip 115 is produced, analyte may be introduced to each of the array recognition complexes 130, and the photochemical changes across the array may be detected and analyzed, producing an even more unique signature that can be used for analyte identification and to distinguish the analyte from chemically or structurally similar species.

The production of chips for attachment of binding moieties is well known in the art. The chip may comprise a Langmuir-Bodgett film, functionalized glass, germanium, silicon, PTFE, polystyrene, gallium arsenide, gold, silver, membrane, nylon, PVP, or any other material known in the art that is capable of having functional groups such as amino, carboxyl, Diels-Alder reactants, thiol or hydroxyl incorporated on its surface. In certain embodiments, these groups may be covalently attached to cross-linking agents so that binding interactions between analyte and recognition complex occur without steric hindrance from the chip surface. Typical cross-linking groups include ethylene glycol oligomer, diamines and amino acids. Any suitable technique useful for immobilizing a recognition complex on a chip is contemplated by this invention, including sialinization. In some embodiments, an organic semiconductor such as DAT may be attached to the chip surface and binding moieties are then attached, covalently or non-covalently, to the DAT.

The array-based chip design 115 may be distinguished from conventional biochips (e.g., U.S. Pat. Nos. 5,861,242, 5,578,832 and 6,071,394) by a number of characteristics, including the use of an organic semiconductor. In certain embodiments of the present invention the affinities of the binding moiety/organic semiconductor couplets for various analytes are unknown at the time they are initially attached to the chip. Target analytes are identified by their pattern of binding to the entire chip, not by their binding to a specific locus on the chip. This system provides greater efficiency and flexibility, in that it is not necessary to prepare binding moieties of known specificity before construction of the chip. Further, previously unknown analytes may be characterized by their pattern of interaction with the chip, without having to clone and sequence their RNA or DNA or prepare high-affinity binding moieties in advance of chip production. In other embodiments, binding moieties with binding specificities for known analytes may be used. In these embodiments, the binding moieties may be attached to the biochip at known locations on the chip. The presence and identity of the analyte are determined by its ability to bind to a discrete site on the biochip.

Embodiments Involving Magnetic Beads

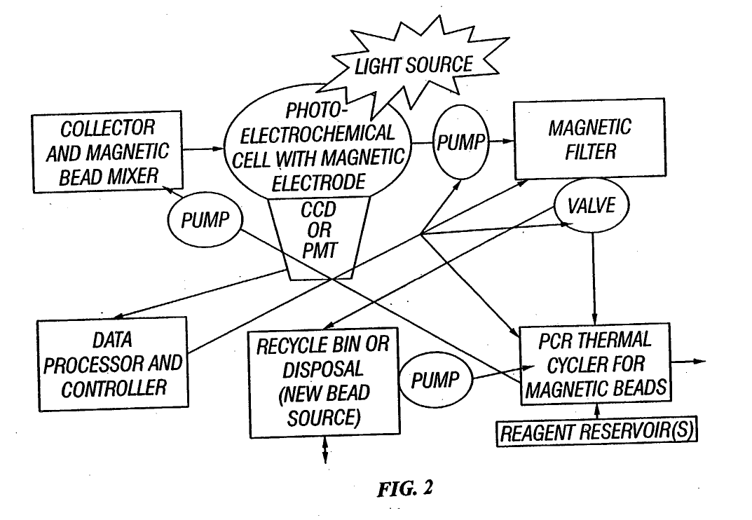

In alternative embodiments, the binding moiety sequences may be attached to magnetic beads instead of to a flat surface. In this case, each recognition complex would comprise a magnetic bead attached to one or more binding moieties. In a preferred embodiment, each binding moiety attached to the same magnetic bead will have the same analyte selectivity. In other embodiments, the binding moiety molecules attached to a single bead may have different selectivities. In certain embodiments, the binding moieties will also be attached to an organic semiconductor, such as DAT. Attachment of binding moieties to DAT would facilitate the detection and quantitation of analyte binding to the binding moieties, as described above. An exemplary recognition complex system utilizing recognition complexes attached to magnetic beads is illustrated in FIG. 2.

The skilled artisan will realize that use of magnetic bead technology would facilitate certain applications of the invention, such as the iterative process for selecting binding moieties of higher specificity and greater binding affinity for the analyte. With magnetic bead technology, the individual recognition complexes are more easily manipulated and separated according to their characteristics. For example, recognition complexes that bind to the analyte may be separated from recognition complexes that do not bind to the analyte by using a magnetic flow cell or filter block, as disclosed in U.S. Pat. No. 5,972,721, incorporated herein by reference in its entirety.

Binding moieties may be synthesized or expressed and attached to magnetic beads. The individual recognition complexes, each corresponding to a magnetic bead attached to one or more binding moieties, together comprise an array, similar to that described above for FIG. 1. The array is added to the magnetic bead mixer and analyte is added and allowed to bind to the binding moieties. The mixture is then transferred to a photochemical cell with a magnetic electrode, where the mixture may be exposed to ultraviolet or other irradiation. A CCD, photomultiplier tube, digital camera or other detection device may be used to obtain absorption or emission spectra. Binding of analyte preferably results in characteristic changes in the photochemical properties of individual recognition complexes. These changes in photochemical properties may be detected and analyzed to produce an analyte signature. Although the suspension of recognition complexes in the bead mixer is random, the use of a magnetic electrode in the photochemical cell provides a spatial distribution of recognition complexes, analogous to the two-dimensional array 115 described above. Beads will deposit and separate on the surface of the magnetic electrode according to their accumulated mass (from binding analyte). This spatial distribution, along with the detected photochemical changes, may be analyzed to produce a unique signature that can be used to identify the analyte.

After detection, the recognition complexes may be transferred to a magnetic filter, where the recognition complexes that bind to the analyte may be separated from those that do not bind analyte. The recognition complexes that do not bind analyte are transferred to the recycle bin, where the binding moieties may be detached from the magnetic beads. The magnetic beads may be disposed of or recycled for attachment to new binding moieties. Those recognition complexes that bind to the analyte attached to magnetic beads are transferred to the magnetic bead mixer for another iteration of the process. This iterative process may be used to select binding moieties that bind with high affinity to the analyte, or may be used to produce an array with greater specificity for the target analyte. In embodiments utilizing nucleic acid binding moieties, an additional amplification step may be added to the iterative process to selectively amplify those nucleic acids that bind to the analyte.

Certain components may be incorporated into a recognition complex system including pumps and valves to facilitate fluid transfer between different components of the recognition complex system. It is anticipated that virtually any pump or valve capable of producing a controlled fluid transfer between one component and another component of the recognition complex system could be used.

Processes for the coupling of molecules to magnetic beads or a magnetite substrate are well known in the art, i.e. U.S. Pat. Nos. 4,695,393, 3,970,518, 4,230,685, and 4,677,055 herein expressly incorporated by reference. Alternatively, DAT may be attached directly to the magnetic bead. Binding moieties may be attached to DAT by electrostatic interaction, hydrogen bonding or other non-covalent interaction. This would facilitate detachment from the DAT/magnetic bead, since the binding moiety would be released, for example, by addition of a solution of the appropriate ionic concentration or pH. Alternatively, the binding moiety may be covalently attached, for example by chemical cross-linking to DAT. A number of potentially chemical cross-linking agents are well known in the art, including EDC, dinitrobenzene, bisimidates, N-hydroxysuccinimide ester of suberic acid, dimethyl-3,3′-dithio-bispropionimidate, 4-(bromoaminoethyl)-2-nitrophenylazide, disuccinimidyl tartarate and azidoglyoxal.

In other embodiments, the organic semiconductor and/or binding moiety may be modified in order to facilitate dissociation between the semiconductor and binding moiety. Such modifications may include the introduction of a cleavage site. Modifications may also include the addition of one or more sulfhydryl groups to permit the formation of disulfide linkages between the semiconductor and binding moiety. Linkers, such as short peptide linkers, may be used to attach the semiconductor to the binding moiety.

The analyte may bind to one or more recognition complexes. Those recognition complexes bound to the analyte may be separated from unbound recognition complexes by mass segregation, using a magnetic filter. The separated binding moieties may be attached to DAT and/or magnetic beads for another iteration of analyte binding and detection, or may be collected and used for other purposes, such as analyte neutralization or preparation of high-affinity diagnostic devices for detecting analyte in the field.

It is envisioned that particles employed in the instant invention may come in a variety of sizes. While large magnetic particles (mean diameter in solution greater than 10 μm) can respond to weak magnetic fields and magnetic field gradients, they tend to settle rapidly, limiting their usefulness for reactions requiring homogeneous conditions. Large particles also have a more limited surface area per weight than smaller particles, so that less material can be coupled to them. In preferred embodiments, the magnetic beads are less than 10 μm in diameter.

Various silane couplings applicable to magnetic beads are discussed in U.S. Pat. No. 3,652,761, incorporated herein by reference. Procedures for silanization known in the art generally differ from each other in the media chosen for the polymerization of silane and its deposition on reactive surfaces. Organic solvents such as toluene (Weetall, 1976), methanol, (U.S. Pat. No. 3,933,997) and chloroform (U.S. Pat. No. 3,652,761) have been used. Silane deposition from aqueous alcohol and aqueous solutions with acid have also been used.

Ferromagnetic materials in general become permanently magnetized in response to magnetic fields. Materials termed “superparamagnetic” experience a force in a magnetic field gradient, but do not become permanently magnetized. Crystals of magnetic iron oxides may be either ferromagnetic or superparamagnetic, depending on the size of the crystals. Superparamagnetic oxides of iron generally result when the crystal is less than about 300 angstroms (Å) in diameter; larger crystals generally have a ferromagnetic character.

Dispersible magnetic iron oxide particles reportedly having 300 Å diameters and surface amine groups are prepared by base precipitation of ferrous chloride and ferric chloride (Fe²⁺/Fe³⁺=1) in the presence of polyethylene imine, according to U.S. Pat. No. 4,267,234. These particles are exposed to a magnetic field three times during preparation and are described as redispersible. The magnetic particles are mixed with a glutaraldehyde suspension polymerization system to form magnetic polyglutaraldehyde microspheres with reported diameters of 0.1 (m. Polyglutaraldehyde microspheres have conjugated aldehyde groups on the surface that can form bonds to amino containing molecules such as binding moieties or DAT.

While a variety of particle sizes are envisioned to be applicable in the disclosed method, in a preferred embodiment, particles are between about 0.1 and about 1.5 μm diameter. Particles with mean diameters in this range can be produced with a surface area as high as about 100 to 150 m²/gm, which provides a high capacity for bioaffinity adsorbent coupling. Magnetic particles of this size range overcome the rapid settling problems of larger particles, but obviate the need for large magnets to generate the magnetic fields and magnetic field gradients required to separate smaller particles. Magnets used to effect separations of the magnetic particles of this invention need only generate magnetic fields between about 100 and about 1000 Oersteds. Such fields can be obtained with permanent magnets that are preferably smaller than the container that holds the dispersion of magnetic particles and thus may be suitable for benchtop use. Although ferromagnetic particles may be useful in certain applications of the invention, particles with superparamagnetic behavior are usually preferred since superparamagnetic particles do not exhibit the magnetic aggregation associated with ferromagnetic particles and permit redispersion and reuse.

The method for preparing the magnetic particles may comprise precipitating metal salts in base to form fine magnetic metal oxide crystals, redispersing and washing the crystals in water and in an electrolyte. Magnetic separations may be used to collect the crystals between washes if the crystals are superparamagnetic. The crystals may then be coated with a material capable of adsorptively or covalently bonding to the metal oxide and bearing functional groups for coupling with binding moieties or DAT.

Embodiments Involving Non-Magnetic Beads, Cells or Particles and Flow Cytometry

In another embodiment, the recognition complexes or analyte of interest may be non-covalently or covalently attached to non-magnetic beads, such as glass, polyacrylamide, polystyrene or latex. Receptor complexes may be attached to such beads by the same techniques discussed above for magnetic beads. After exposure of analyte to receptor complexes, those complexes bound to analyte may be separated from unbound complexes by flow cytometry. Non-limiting examples of flow cytometry methods are disclosed in Betz et al. (1984), Wilson et al. (1988), Scillian et al. (1989), Frengen et al. (1994), Griffith et al. (1996), Stuart et al. (1998) and U.S. Pat. Nos. 5,853,984 and 5,948,627, each incorporated herein by reference in its entirety. U.S. Pat. Nos. 4,727,020, 4,704,891 and 4,599,307, incorporated herein by reference, describe the arrangement of the components comprising a flow cytometer and the general principles of its use.

In the flow cytometer, beads, cells or other particles are passed substantially one at a time through a detector, where each particle is exposed to an energy source. The energy source generally provides excitatory light of a single wavelength. The detector comprises a light collection unit, such as photomultiplier tubes or a charge coupled device, which may be attached to a data analyzer such as a computer. The beads, cells or particles can be characterized by their response to excitatory light, for example by detecting and/or quantifying the amount of fluorescent light emitted in response to the excitatory light. Changes in size due to binding of analyte to binding moiety can also be incorporated into sorting strategies. Beads or cells exhibiting a particular characteristic can be sorted using an attached cell sorter, such as the FACS Vantage™ cell sorter sold by Becton Dickinson Immunocytometry Systems (San Jose, Calif.).

That system is well suited to use with an organic semiconductor, such as DAT, that has fluorescent and luminescent properties. Using a flow cytometer, it is possible to separate beads, cells or particles that are associated with recognition complexes bound to analytes, from unbound complexes, by detecting the presence of and characterizing the photochemical properties of the organic semiconductor. Because those properties change upon binding of recognition complex to analyte, it is possible to separate bead-attached recognition complexes that bind to analyte from complexes that do not bind analyte. This process is even simpler when the analyte is incorporated into a cell or cell fragment, or attached to a bead. In this case, only analytes bound to recognition complexes should show a fluorescent or other spectroscopic signature associated with the organic semiconductor.

Flow cytometry may be used to purify or partially purify analytes that bind to a particular binding moiety, or to purify or partially purify binding moieties that bind to a particular analyte. Other manipulations may include sorting for differences in fluorescence and/or size that represent differences in binding affinity or avidity of analyte for binding moiety or the number of binding moieties bound to each analyte or of analyte bound to each binding moiety.

Embodiments Involving Flow Cells

In another exemplary embodiment, each of the recognition complexes associated with the array 115 may comprise a flow cell. The flow cell is designed to be easily removable from the array 115 and to sit directly on an inverted optical microscope. Either transmitted or incident illumination may be used since the flow cell is transparent. The primary purpose for implementing the array 115 using flow cells is to permit more detailed analysis of the analyte and binding moiety interaction with particulate structures.

Recognition Complex System Model

In another embodiment, the binding moiety sequences that exhibit the greatest degree of affinity for the analyte can be expressed and/or chemically synthesized and employed as an agent to neutralize adverse biological effects associated with the detected analyte. The recognition complex system initially generates a somewhat non-specific response. Following one or more rounds of selection of binding moiety sequences that bind to analyte with higher affinity, the recognition complex system responds in a more specific way to neutralize known or previously unknown analytes.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of DAT and Spectroscopic Characteristics

Synthesis

DAT was synthesized as disclosed herein. 3-Amino-L-tyrosine (1:776 gm) was dissolved in 50 ml of distilled water. NaNO₂ (0.417 gm) was added to the solution. After 4 min, the mixture of 3-AT and sodium nitrite was subjected to refluxing for approximately 8 hours. The resulting DAT was precipitated by addition of acetone and the precipitate was allowed to sit overnight in a separatory funnel.

DAT was collected from solution by centrifugation at 3,000 rpm for 10 min. DAT was resuspended in distilled water and dialyzed against distilled water in a 3,500 Dalton molecular weight cutoff bag.

Spectroscopic Characteristics

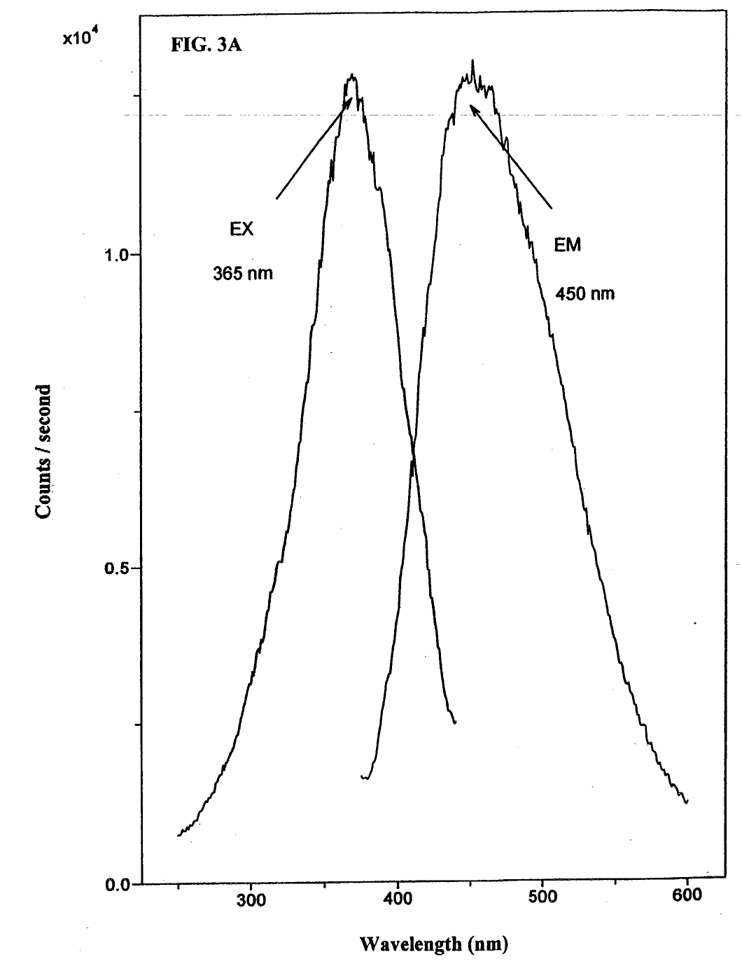

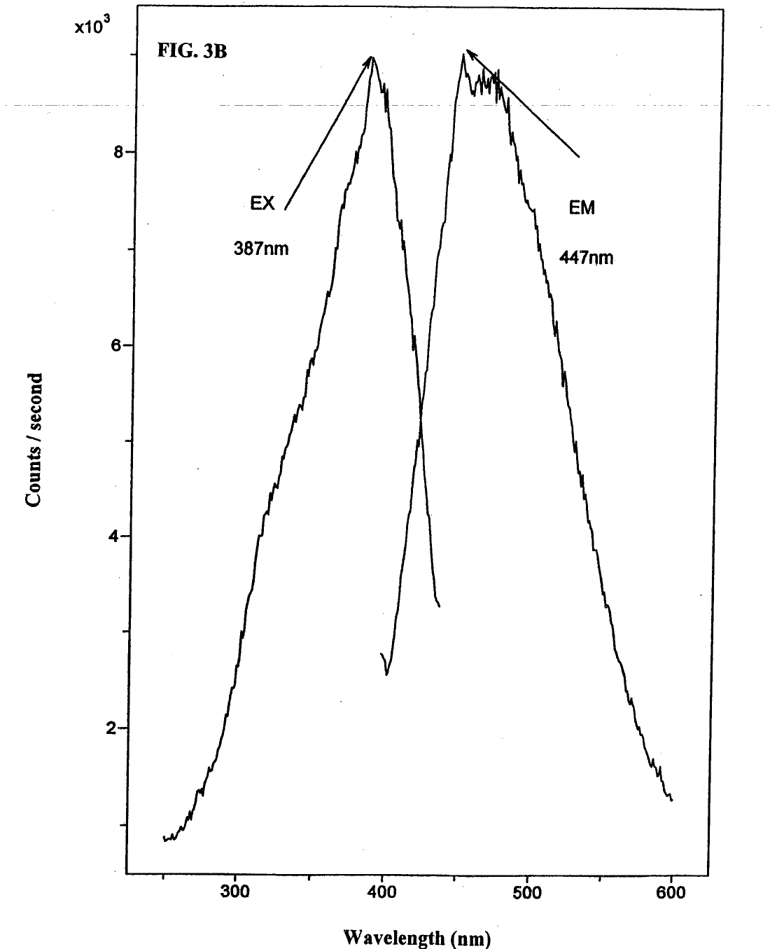

The spectroscopic properties of DAT were compared to those of DALM in a NaBr solvent system (FIGS. 3A-3B). The fluorescent properties of DALM (0.12 mg/ml) (FIG. 3A) and DAT (0.132 mg/ml) (FIG. 3B) were similar. Under these conditions DALM exhibited an excitation peak at 365 nm and an emission peak at 450 nm (FIG. 3A), while DAT exhibited an excitation peak at 387 nm and an emission peak at 447 nm.

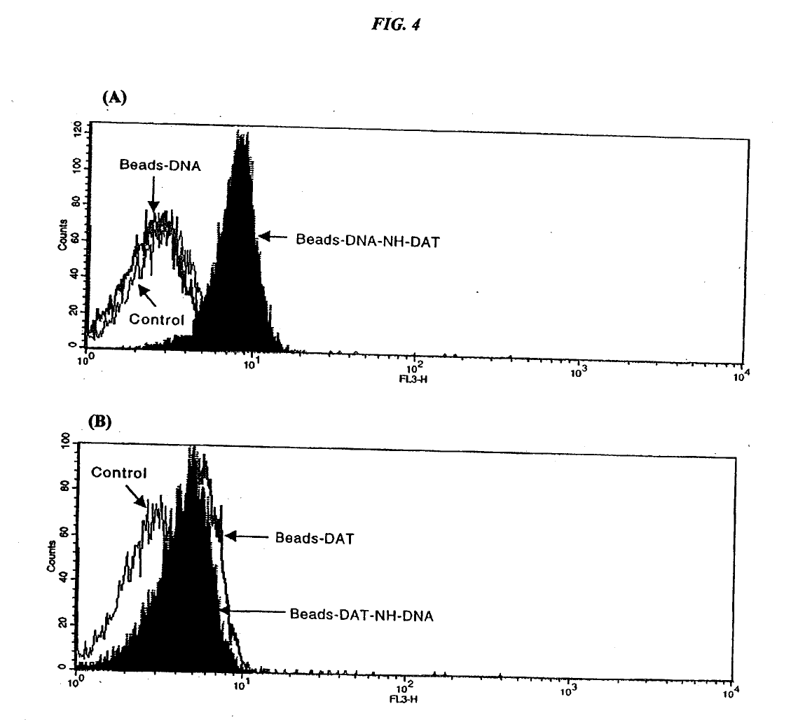

The fluorescent properties of DALM are affected by interaction with ligands, such as DNA, allowing the spectroscopic detection of analyte binding by organic semiconductor fluorescence (U.S. Pat. No. 6,303,316). FIG. 4A and FIG. 4B show that the spectroscopic properties of DAT are also affected by ligand interaction. FIG. 4 shows the fluorescence intensity (y-axis) of DAT and/or DNA conjugated to beads. As shown in FIG. 4A, the addition of DAT to magnetic beads conjugated to DNA results in a large increase in fluorescence intensity. FIG. 4B shows that addition of DNA to beads conjugated to DAT results in a smaller, but detectable shift in fluorescence intensity. In this case, it appears that DNA acts at least in part to quench DAT fluorescence. Thus, the spectroscopic properties of DAT are dependent on ligand interaction, as previously shown for DALM (U.S. Pat. No. 6,303,316).

### Example 2

### DNA Based Recognition Complex System

Oligonucleotides may be obtained from commercial sources, such as Ransom Hill Biosciences, Sigma Chemical Co., or Genosys Corp. DAT is synthesized as described in Example 1 above. All polymerase chain reaction (PCR) reagents, including dideoxynucleotides, are from commercial sources (e.g., Promega, Boehringer-Mannheim). Binding buffer (BB) is composed of 0.5 M NaCl, 10 mM Tris-HCl, and 1 mM MgCl₂ in deionized water (pH 7.5 to 7.6).

Various types of arrays of nucleic acid ligands may be generated. In a ligated array, nucleic acid ligand diversity may be increased compared to the starting random oligomers, by truncating longer chains with the addition of dideoxynuclotides during a PCR step and covalently linking non-contiguous DNA chains together with Taq DNA ligase.

The PCR chain termination step involves addition of 6.6 μg of random 60 mers as a self-priming (due to partial hybridization) PCR template with 8 μl of each deoxy/dideoxynucleotide (i.e., d/ddA, d/ddC, d/ddG, d/ddT) and 20 μl (80 units) of Taq polymerase per tube. The tubes are PCR amplified using the following temperature profile: 96° C. for 5 min, followed by 40 cycles of 96° C. for 1 min, 25° C. for 1 min 72° C. for 1 min. PCR extension is completed at 72° C. for 7 min and tubes are stored at 4 to 6° C. until used. The collection of nucleic acid ligand species present as overlapping random 60 mers or as ligated and truncated DNAs constitutes a library of nucleic acid ligands.

An exemplary method for attaching an array of binding moiety-organic semiconductor couplets to glass or other solid surfaces involves direct attachment of DAT to the surface. Nucleic acids or other binding moieties may be attached to DAT using non-covalent interactions or by covalent or other attachment techniques known in the art. Glass slides are cleaned with alcoholic potassium hydroxide, washed with DI (deionized water) and dried overnight. To approximately 150 ml of acetone is added 8 ml of water and 12 ml of 3-aminopropyltriethoxysilane. Acetone is added to a final volume of 200 ml. The slides are placed on the bottom of a rectangular plastic storage container and the acetone solution is poured over them. After two hours at room temperature on an orbital shaker (75 rpm) the slides are washed twice with acetone.

DAT may be covalently attached to the amino groups on the surface of the glass. Reduced synthetic DAT is dissolved in 2 to 3 ml of 0.1 M NaOH. 0.1 M MOPS buffer (pH 7) is added to a final volume of 50 ml. The DAT solution is poured over the glass slides in the storage container. Additional MOPS buffer is added until all slides are completely covered. EDC (N,N-(3-dimethylaminopropyl)-N′-ethyl-carbodiimide hydrochloride) is dissolved in MOPS buffer and immediately added to the slides, while shaking on an orbital shaker. This addition is repeated every 15 min for an additional four times. After another hour, more EDC is added. The slides are incubated at room temperature for another two hours with shaking, then rinsed and dried overnight. DAT is covalently attached to the glass slides. Although glass is used in this example, the skilled artisan will realize that any solid surface capable of being coated with 3-aminopropyltriethoxysilane or an equivalent linker compound could be used in the practice of the invention.

The fluorescence emission spectra of DAT before and after interaction with random 60 mer DNA may be compared. Excitation is performed at 390 nm. The fluorescence of DAT with and without added nucleic acids shows enhanced fluorescence intensity and an emission spectrum shift of DAT after binding DNA, demonstrating a fluorescence energy transfer from DAT to bound DNA.

### Example 3

### Interaction of Recognition Complex System with Cholera Toxin

Randomized 40 mer template DNA flanked by 5′ polyA and 3′ polyT (10 mer) regions is obtained from Genosys Corp. and PCR amplified in the presence of ddNTPs and 2 units of Taq ligase. Cholera toxin is obtained from Sigma Chemical Co. (St. Louis, Mo.). Ten μl of PCR product per gel lane is mixed 1:1 with DNA loading buffer and electrophoresed at 100 V in 10% polyacrylamide precast minigels in TBE. Gels are then treated with DAT and/or cholera toxin in 1× binding buffer (BB). Gel lanes are cut and separated and scanned for fluorescence intensity at 260 nm excitation and 420 nm emission, using a Perkin-Elmer LS-50B spectrofluorometer and fiber optic plate reader attached in the TLC plate mode. The gel lanes are scanned before and after the addition of analyte (0.1 mg/ml of cholera toxin for 1 hr. at ambient temperature with mixing).

Differences in spatial fluorescence patterns are seen for nucleic acid ligand arrays in polyacrylamide gels with 0.1 mg/ml whole cholera toxin with and without DAT augmentation. Addition of DAT primarily amplifies the low-level fluorescence of the DNA and changes the spatial fluorescence characteristics.

### Example 4

### Production of Nucleic Acid Ligands with High Affinity for Anthrax

Systematic Evolution of Ligands by EXponential enrichment (SELEX) is used to select and PCR amplify nucleic acid ligands capable of binding to and detecting nonpathogenic Sterne strain Bacillus anthracis spores. A simplified affinity separation approach is employed, in which autoclaved anthrax spores are used as the separation matrix.

Primers and Templates—The SELEX technique is used to amplify and select for analyte-binding nucleic acid ligands, using whole anthrax spores as the analyte. Primers and two sets of templates are designed to simplify PCR amplification by utilizing mirrored ends to allow amplification of the nascent strand using a single type of free primer (Bruno, 1997). Both templates consist of 60 mers. These are composed of 5′-poly A and 3′ poly T 10 mers, sandwiching a random 40 mer. One set of DNA molecules (hereafter the “capture” set) consist of templates with an amino-six carbon linker (NH₂-C6) attached to their 5′ ends for conjugation to tosyl-activated magnetic microbeads (M-280; 2.8 μm diameter, Dynal Corp., Lake Success, N.Y.), and free unlabeled poly A 10 mer primers. The other DNA set (hereafter the “reporter” set) is identical to the capture set, except that both the template and the primer are 5′-biotinylated to afford detection by binding of labeled avidin.

All oligonucleotides are obtained from Ransom Hill Biosciences, Inc. (Ramona, Calif.). All PCR reagents, except Taq polymerase, are obtained from Perkin-Elmer Corp. Taq polymerase is obtained from Fisher Scientific Corp. (Pittsburgh, Pa.).

Anthrax Spores—Sterne strain veterinary vaccine anthrax spores (Thraxol-2, Mobay Corp., Shawnee, Kans.) are streaked onto blood agar plates and incubated at 37° C. for 5 days to promote extensive sporulation and autolysis of vegetative cells. Colonies are gently washed and scraped from blood agar plates into 10 ml of filter-sterilized deionized water. Spores are resuspended in 50 ml of filter-sterilized deionized water and autoclaved at 134° C. for 60 min to produce a dead stock spore suspension used in nucleic acid ligand development and detection assays. Stock spore suspension concentration is determined by the average of hemocytometer counts using phase-contrast microscopy at 600× magnification.

Detection—Streptavidin (Southern Biotechnology Associates Inc., Birmingham, Ala.) is labeled with N-hydroxy-succinimide-Ru(bpy)₃²⁺ (IGEN International Inc., Gaithersburg, Md.) in a 15:1 protein to N-hydroxysuccinimide ECL label molar ratio as described by Gatto-Menking et al. (1995). Avidin-biotin complex reagent from a “Vectastain Elite ABC”—peroxidase kit is from Vector Laboratories, Inc. (Burlingame, Calif.). ABTS (2,2′-azino-di(3-ethyl-benzthiazoline-6-sulphonic acid) is obtained as a mixture with H₂O₂ added from Kirkegaard and Perry Laboratories (Gaithersburg, Md.) for colorimetric detection of spore-bound biotinylated nucleic acid ligands.

PCR Amplification—PCR is carried out prior to exposure of the nucleic acid ligand library to anthrax spores to optimize the annealing temperature. A 600 μl PCR master mix consists of 1 ng of either capture or reporter DNA templates, 1 μM final concentration of appropriate primer, 10 mM of each deoxynucleotide, 5 mM MgCl₂, 10 mM Tris-HCl, 50 mM KCl and 50 units of Taq polymerase in autoclaved, deionized water. PCR conditions are: initial denaturation at 96° C. for 5 min; 40 cycles of 96° C. for 1 min, 47° C. for 1 min, 72° C. for 1 min; and final extension at 72° C. for 7 min.

SELEX Procedures—A DNA to spore ratio of 10,256 ng DNA/10⁶ spores is used. The method involves immediate addition of hot (96° C.) DNA (either capture or reporter templates) to 6.5×10⁶ anthrax spores in 400 μl of sterile 2× binding buffer (2× BB, 1M NaCl, 40 mM Tris-HCl and 2 mM MgCl₂ in autoclaved, deionized water, pH 7.5-7.6), (Ellington & Szostak,1990; Bruno, 1997) at ambient temperature with immediate mixing for 1 h. Spore suspensions are pelleted by centrifugation at 9,300×g for 10 min. Spores bound to nucleic acid ligands are resuspended in 1 ml of sterile 1× BB at room temperature. Spores are pelleted and washed twice more in 1× BB.

The spore pellet is overlaid with 100 μl of 1× BB and heated to 96° C. for 5 min to heat-liberate the bound nucleic acid ligands. The hot supernatant (100 μl) is siphoned from the spore pellet and 90 μl of the supernatant is PCR amplified. The remaining 10 μl of hot supernatant is electrophoresed in 2% agarose at 80 V in cold 1× Tris-borate-EDTA (TBE) buffer for 30 min. Gels are stained in 0.5 μg/ml of ethidium bromide in 1× TBE for 10 min followed by a 30 min wash in deionized water. Four rounds of SELEX are performed. Fresh aliquots of the stock spore suspension are used for each round.

Nucleic acid ligand-magnetic bead preparation. Capture nucleic acid ligands (100 μl of round four PCR product) are conjugated to 400 μl of stock tosyl-activated Dynal M-280 magnetic beads (approximately 2.6×10⁸ beads) in the presence of 1 ml of sterile 50 μM sodium borate (pH 9.5). Conjugation is performed for 2 h at 37° C. with periodic agitation, followed by additional coupling overnight at 4° C. Magnetic microbeads are collected for 10 min using a Corning Corp. (Corning, N.Y.) magnetic separator (60 tube capacity model). Beads are washed once in 3 ml of sterile 1× BB and resuspended in 2 ml of sterile 1% bovine serum albumin (BSA), 50 μM sodium borate buffer for 2 h at 37° C. to neutralize any unreacted tosyl groups. Beads are washed three times in 3 ml of 1× BB and resuspended in 2 ml of 1× BB. The stock nucleic acid ligand-magnetic bead suspension is stored at 4° C. until used in ECL assays.

Colorimetric detection of nucleic acid ligand binding to spores is achieved by addition of 200 μl of Vectastain Elite avidin-biotin complex (ABC)-peroxidase reagent in 1× BB to resuspended spore pellets from each round of SELEX (including a pre-SELEX control). After 30 min at ambient temperature, spores are centrifuged and washed three times in 1 ml of 1× BB. Spore pellets are resuspended in 400 μl of ABTS for 15 min. Four 100 μl aliquots from each tube are placed into microtiter wells and absorbance at 405 nm is determined using an automated plate reader.

ECL-based and colorimetric binding assays are employed to assess nucleic acid ligand binding to anthrax spores. Decreasing levels of PCR products as a function of SELEX round show that tighter binding nucleic acid ligands are selected after each round. Using these methods, nucleic acid ligands with high affinity for target analytes may be generated.

### Example 5

### Neutralization of Biohazardous Agents Using Organic Semiconductors

In certain embodiments, organic semiconductors may be used to neutralize biohazardous agents, such as viruses, microbes, spores or potentially single molecules. Organic semiconductors activated, for example, by hydrogen peroxide and bicarbonate and pulsed with microwave radiation act as photochemical transducers, releasing an intense pulse of visible light. High power pulsed microwave radiation (HPM), applied to solutions containing dissolved carbon dioxide (or bicarbonate), hydrogen peroxide and an organic semiconductor generates sound, pulsed luminescence and electrical discharge. Microbes exposed to these conditions experience damage comparable to short time, high temperature insults, even though measurable localized temperatures are insufficient to cause the observed effects.

i Bacillus anthracis spores are incubated with DAT and exposed to a high power microwave (HPM) pulse. Bacillus anthracis (BA; Sterne strain) spore vaccine (Thraxol™, Mobay Corp., Animal Health Division, Shawnee, Kans. 66201) is centrifuged, the supernatant decanted and the button washed with chilled deionized water. Dilute powdered milk solution is made to a concentration of 25 mg of powdered milk solids/ml of deionized water, filtered through a 0.2 micron filter. The BA button is resuspended in 1 ml of sterile milk solution to form a BA suspension.

For pulsed microwave exposure, 0.5 ml of BA spore suspension is placed into 0.2 micron-filter centrifuge tubes (Microfilterfuge™, Rainin Instrument Co., Inc., Woburn, Mass. 01888-4026). The spores are centrifuged onto the filter at 16,000×g for 15 min. Tubes are refilled with 1.5 ml of a reaction mixture consisting of 0.9 ml saturated sodium bicarbonate solution, 0.1 ml of 1:10 DAT, and 0.33 ml 3% hydrogen peroxide.

The filter, with the BA spores, is inserted into the tube to a level just below the meniscus of the fluid. The solution is exposed to 10 pulses per second of HPM (1.25 GHz, 6 μs pulse, 2 MW peak incident power), starting at 3 minutes and 22 seconds after placing the reaction mixture in front of the microwave waveguide. The exposure lasts for 13 min and 28 sec. Total radiation exposure is for 48 msec.

Control spores exposed to HPM in the absence of DAT remain intact. Anthrax spores exposed to HPM in the presence of DAT lyse. In alternative embodiments, DAT may be attached to binding moieties with affinity for an analyte before exposure to activating radiation, resulting in an increased efficiency of analyte neutralization.

### Example 6

### Neutralizing Anthrax Spores with DAT

Materials and Methods

Anthrax spores preincubated with organic semiconductors were exposed to microwave radiation as disclosed in Example 5, with the following modifications. Anthrax spores pre-treated with organic semiconductor were applied to No. 3 Whatman filters contained in snap-lid petri dishes. The dishes were arranged in a nine plate array. Dishes were centered vertically and horizontally in front of a 2.06 GHz L-band microwave transmitter and exposed to microwave radiation at 400 W, 10 Hz with 10 msec pulses for 15 min exposure time. After microwave exposure, filter papers were vigorously vortexed in buffer and aliquots were plated to determine colony forming units (CFU). The percentage of kill was calculated as [1−(test CFU/control CFU)]×100.

The efficacy of DAT in promoting microwave induced killing of anthrax spores was examined. In some cases, purified DAT was treated with hydrogen peroxide to produce oxidized DAT (O-DAT).

Results

The oxidized form (O-DAT) was more efficient at inducing anthrax spore destruction than the unoxidized form. Percent kill observed was 45.7% for O-DAT. Under the conditions of this study, pretreatment with unoxidized DAT did not result in detectable destruction of anthrax spores.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

Betz et al., Cytometry 5: 145-150, 1984.

Bruno and Yu, Immunomagnetic-electrochemiluminescent detection of Bacillus anthracis spores in soil matrices. Appl. Environ. Microbiol. 62: 3474-76, 1996.

Bruno, In vitro selection of DNA to chloroaromatics using magnetic microbead-based affinity separation and fluorescence detection. Biochim. Biophys. Res. Comm. 234, 117-120, 1997.

Bruno et al., Preliminary electrochemiluminescence studies of metal ion-bacterial diazoluminomelanin (DALM) interactions. J. Biolumin. Chemilumin. 13: 117-123, 1998.

Ellington and Szostak, In vitro selection of RNA molecules that bind specific ligands. Nature 346: 818-822, 1990.

Frengen et al., Clin. Chem. 40/3: 420-425, 1994.

Gatto-Menking et al., Sensitive detection of biotoxoids and bacterial spores using an immunomagnetic electrochemiluminescence sensor. Biosensors Bioelectronics 10: 501-507, 1995.

Griffith et al., Cytometry 25: 133-143, 1996.

Kiel et al. “Luminescent radio frequency radiation dosimetry.” Bioelectromagnetics 20:46-51, 1999a.

Kiel et al., “Pulsed microwave induced light, sound, and electrical discharge enhanced by a biopolymer.” Bioelectromagnetics 20:216-223, 1999b.

Scillian et al., Blood 73: 2041-2048, 1989.

Stuart et al., Cytometry 33: 414-419, 1998.

Weetall, H. W. in: Methods in Enzymology, K. Mosbach (ed.), 44:134-148, 140, 1976

Wilson et al., J. Immunol. Methods 107: 231-237, 1988.

U.S. Pat. No. 3,652,761

U.S. Pat. No. 3,970,518

U.S. Pat. No. 3,933,997

U.S. Pat. No. 4,230,685

U.S. Pat. No. 4,267,234

U.S. Pat. No. 4,599,307

U.S. Pat. No. 4,677,055

U.S. Pat. No. 4,695,393

U.S. Pat. No. 4,704,891

U.S. Pat. No. 4,727,020

U.S. Pat. No. 5,003,050

U.S. Pat. No. 5,156,971

U.S. Pat. No. 5,578,832

U.S. Pat. No. 5,853,984

U.S. Pat. No. 5,856,108

U.S. Pat. No. 5,861,242

U.S. Pat. No.5,902,728

U.S. Pat. No.5,948,627

U.S. Pat. No. 5,972,721

U.S. Pat. No. 6,071,394

U.S. Pat. No. 6,303,316

What is claimed is:

1. A composition comprising polydiazoaminotyrosine (DAT).

2. The composition of claim 1, wherein the DAT is operably coupled to at least one binding moiety.

3. The composition of claim 2, wherein the binding moiety is a protein, a peptide, an antibody, an antibody fragment or a nucleic acid ligand.

4. The composition of claim 3, further comprising two or more binding moieties.

5. The composition of claim 4, wherein the binding moieties are identical.

6. The composition of claim 4, wherein the binding moieties are selective for different analytes.

7. The composition of claim 1, wherein the DAT is in an oxidized form.

8. A recognition complex system comprising two or more recognition complexes, each recognition complex comprising DAT operably coupled to a binding moiety.

9. The recognition complex system of claim 8, wherein the recognition complexes are attached to a surface.

10. The recognition complex system of claim 9, wherein the surface is selected from the group consisting of magnetic beads, glass beads, plastic beads, a planar surface, a chip, a badge, a card and a flow cell.

11. A method for obtaining one or more binding moieties that bind with high affinity for an analyte comprising the steps of:
a) generating multiple recognition complexes, each recognition complex comprising a binding moiety operably coupled to DAT;
b) contacting the recognition complexes with the analyte;
c) separating those recognition complexes that bind to the analyte from those recognition complexes that do not bind to the analyte; and
d) repeating (b) and (c) until one or more binding moieties that bind with high affinity to the analyte are obtained.

12. The method of claim 11, wherein the binding moiety is a nucleic acid ligand.

13. The method of claim 12, further comprising amplifying those nucleic acid ligands that bind to the analyte.

14. The method of claim 11, wherein the recognition complexes are attached to magnetic beads.

15. The method of claim 14, wherein said separating uses a magnetic flow cell, wherein beads attached to recognition complexes that bind to the analyte are separated in the flow cell from beads attached to recognition complexes that do not bind to the analyte.

16. A method for neutralizing an infectious agent comprising the steps of:
a) contacting the infectious agent with DAT; and
b) activating the DAT.

17. The method of claim 16, wherein the activating comprises exposing the DAT to sunlight, heat, laser radiation, ultraviolet radiation, infrared radiation, radiofrequency radiation, pulsed corona radiation or microwave radiation.

18. The method of claim 17, further comprising (i) obtaining one or more binding moieties that bind to the infectious agent; and (ii) forming DAT:binding moiety couplets.

19. The method of claim 16, wherein the infectious agent is an anthrax spore.

20. A method of producing DAT comprising
a) obtaining a solution of 3-amino-L-tyrosine (3-AT); and
b) contacting the 3-AT with an alkali metal nitrite.

21. The method of claim 20, wherein the alkali metal nitrite is sodium nitrite.

22. The method of claim 21, further comprising refluxing the solution containing 3-AT and sodium nitrite.

23. The method of claim 22, further comprising precipitating the DAT by addition of acetone.

24. The method of claim 23, further comprising collecting the precipitated DAT.

25. The method of claim 24, wherein the precipitated DAT is collected by centrifugation.

26. The method of claim 22, wherein said refluxing occurs for 8 hours.

27. A composition comprising DAT, wherein the DAT is made by the method of claim 20.
